(19) United States
(12) Patent Application Publication
(10) Pub. No.: US 2007/0073558 A1
(43) Pub. Date: Mar. 29, 2007

(54) Internet based patient-monitoring system featuring interactive messaging engine

(75) Inventors: Christopher Hall, San Francisco, CA (US); Vance Whitson Lanier JR., San Francisco, CA (US); Matthew Banet, Del Mar, CA (US); Randon Schultz, Venice, CA (US); Marshal Dhillon, San Diego, CA (US); Adam Fleming, San Diego, CA (US); Henk Visser, San Diego, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Berkeley HeartLab,Inc.

(21) Appl. No.: 11/522,562

(22) Filed: Sep. 18, 2006

Related U.S. Application Data
(60) Provisional application No. 60/721,756, filed on Sep. 29, 2005.

(51) Int. Cl. G06Q 10/00; A61B 5/00

(52) U.S. Cl. 705/2; 600/300

(57) ABSTRACT

The invention provides: 1) a monitoring device featuring systems that monitor the patient's vital sign and exercise information; 2) an Internet-based system configured to receive, store, and display vital sign and exercise from the monitoring device, and blood test information from an external blood test; and 3) a messaging system configured to process the vital sign, exercise, and blood test information, and in response send a personalized message to the patient.

### CROSS REFERENCES TO RELATED APPLICATION

This application claims the benefit of U.S. Provisional Patent Application Ser. No. 60/721,756 filed Sep. 29, 2006 and is hereby incorporated by reference.

### BACKGROUND

1. Field of the Invention The present invention relates to a patient-monitoring system that processes information, e.g. information describing a patient's medical history, vital signs, and blood chemistry, and in response sends out a personalized message to the patient.

2. Description of the Related Art

Although mortality rates for cardiovascular disease (CVD) have been declining in recent years, this condition remains the primary cause of death and disability in the United States for both men and women. Currently CVD affects approximately 12 million Americans. Atherosclerotic cardiovascular disease (ASCVD), a form of CVD, can cause hardening and narrowing of the arteries, which in turn restricts blood flow and impedes delivery of vital oxygen and nutrients to the heart. This can lead to coronary heart, cerebral vascular, and peripheral vascular diseases, and results in approximately 75% of all deaths attributed to CVD.

Elevated concentrations of low-density lipoprotein cholesterol (LDL cholesterol) are causally related to the onset of ASCVD because over time these compounds contribute to a harmful formation of plaque on an artery's inner walls, thereby restricting LDL cholesterol, which is therefore typically referred to as ‘bad cholesterol’. Conversely, high-density lipoprotein cholesterol (HDL cholesterol) can bind with LDL cholesterol in the bloodstream and transport it to the liver for disposal. Because of this process, called ‘reverse cholesterol transport’, a high level of HDL cholesterol appears to lower a patient's risk of developing heart disease and stroke. HDL cholesterol is therefore typically referred to as ‘good cholesterol’.

A lipoprotein analysis (also called a lipoprotein profile or lipid panel) is a blood test that measures, among other compounds, blood levels of total cholesterol, LDL cholesterol, and HDL cholesterol. One method for measuring HDL and LDL cholesterol is described in U.S. Pat. No. 6,812,033, entitled ‘Method for identifying risk cardiovascular disease patients’. This patent, assigned to Berkeley HeartLab Inc. and incorporated herein by reference, describes a blood test based on a gradient-gel electrophoresis (GGE). Gradient gels used in GGE are typically prepared with varying concentrations of acrylamide and can separate macromolecules with relatively high resolution compared to conventional electrophoretic gels. Sub-classes of both HDL and LDL cholesterol can be determined by GGE. For example, GGE can differentiate up to seven subclasses of LDL cholesterol (classified as LDL I, IIa, IIb, IIIa, IIIb, IVa, and IVb), and up to five subclasses of HDL (classified as HDL 2b, 2a, 3a, 3b, 3c). These tests correlate to a technique called analytic ultracentrifugation (AnUC), which is an established clinical research standard for lipoprotein subfractionation.

GGE can differentiate the most atherogenic particles, LDL IIIa, IIIb, and IVb, and also the most helpful HDL particle, HDL 2b. Elevated levels of LDL IVb, which represents the smallest LDL cholesterol particles, have been reported to have an independent association with arteriographic progression; a combined distribution of LDL IIIa and LDL IIIb typically reflects the severity of this trait. High levels of HDL 2b increase the efficacy of reverse cholesterol transport, while low levels of HDL 2b can increase the risk of CVD.

GGE can be combined with other blood tests to collectively measure the following compounds:

Total Cholesterol

LDL Cholesterol (and subclass distribution)

HDL Cholesterol (and subclass distribution)

Triglycerides

Apo B-Particle

Apo B Ultra Particle

Lipoprotein

Apo E Genotype

Fibrinogen

Folate

HbA_{1c}

C-Reactive Protein

Homocysteine

Glucose

Insulin

Chlamydia

Other Compounds

Elevated blood pressure is another significant risk factor for CVD. The relationship between blood pressure and the risk of CVD is typically continuous, consistent and independent of other risk factors. For example, each increment of 20 mmHg for systolic blood pressure and 10 mmHg for diastolic blood pressure doubles the risk of CVD across the entire blood pressure range, starting with a pressure of 115/75 mm Hg. Lifestyle modifications, such as weight loss, diets that reduce sodium and fat, smoking cessation, increase in aerobic activity, and reduction in alcohol intake, can lower blood pressure, and thus reduce the risk of CVD.

### SUMMARY OF THE INVENTION

The present invention provides an Internet-based system featuring a messaging engine that sends personalized text messages to a patient's email address, cell phone, or monitoring device. Messages include patient-specific content (e.g., treatment plans, diet recommendations, educational content) that helps drive the patient's compliance in a disease-management program (e.g. a cardiovascular risk reduction program), motivate the patient to meet predetermined goals and milestones, and encourage the patient to schedule follow-on medical appointments.

To generate personalized messages the messaging engine processes the following information, each of which is defined in more detail: 1) information from one or more blood tests; 2) vital sign information; 3) exercise information; and 4) personal information. The messaging engine is bi-directional, meaning it sends personalized messages to the patient after processing the above-mentioned information, and in addition receives and processes messages sent from the patient. ‘Blood test information’, as used herein, means information collected from one or more blood tests, such as a GGE test. Blood test information can include concentration, density, amounts, or any other information describing blood-borne compounds, including but not limited to total cholesterol, LDL cholesterol (and subclass distribution), HDL cholesterol (and subclass distribution), triglycerides, Apo B particle, Apo B ultra particle, lipoprotein, Apo E genotype, fibrinogen, folate, HbA_{1c}, C-reactive protein, homocysteine, glucose, insulin, chlamydia, and other compounds. ‘Vital sign information’, as used herein, means information collected from patient using a medical device that monitors the patient's cardiovascular system. This information includes but is not limited to heart rate (measured at rest and during exercise), blood pressure (systolic, diastolic, and pulse pressure), blood pressure waveform, pulse oximetry, optical plethysmograph, electrical impedance plethysmograph, stroke volume, ECG and EKG, temperature, weight, percent body fat, and other properties. ‘Exercise information’, as used herein, means information that characterizes a patient's exercise habits, including but not limited to steps, miles run or biked, duration of any type of exercise, degree of exertion during exercise, calories burned during exercise, and heart rate and other cardiovascular information measured during exercise. ‘Personal information’, as used herein, means information such as age, gender, medical history, ethnicity, current medications, and other information that can be used alone or in combination with the above-mentioned properties to, among other things, develop metabolic and cardiovascular risk profiles to diagnose and manage a patient.

Specifically, in one aspect, the invention provides: 1) a monitoring device featuring systems that monitor the patient's vital sign and exercise information; 2) an Internet-based system configured to receive, store, and display vital sign and exercise information from the monitoring device, and blood test information from an external blood test; and 3) a messaging system configured to process the vital sign, exercise, and blood test information, and in response send a personalized message to the patient. These systems interconnect and operate in concert to drive a constructive, personalized interaction between a medical professional and the patient. Ultimately the invention forms an effective tool that drives compliance and reduces risk that a patient's CVD progresses.

In embodiments, the messaging system is further configured to send a message to the patient's email address, wireless device, or monitoring device. The message is typically a text message, SMS message, HTML-based message, or other form of message that propagates over an http, https, or wireless protocol. Messages typically reference the patient's vital sign, exercise, and blood test information, as described in more detail below. In addition, messages can include a variety of content, including diet and exercise recommendations, recipes, program goals, progress toward goals, articles, educational content, links to web pages, or related content.

In other embodiments, the messaging system is additionally configured to receive messages from the patient. For example, the messaging system can send a message that includes a text field wherein the patient can enter a response. In this case, the Internet-based system typically includes a software component that parses the response entered in the text field once the message is received.

In a particular embodiment, the database stores blood test information measured from a GGE-based blood test, taken alone or combined with other blood tests. Such tests are described in U.S. Pat. 5,925,229, entitled “Low density lipoprotein fraction assay for cardiac disease risk” and U.S. Pat. No. 6,576,471, entitled “Methods, systems, and computer program products for analyzing and presenting NMR lipoprotein-based risk assessment results”, the contents of which are incorporated herein by reference.

The Internet-based system typically features a website with one or more web pages that display the blood test, vital sign, exercise, and personal information. In embodiments, the website includes a first web interface that displays information for a single patient, and a second web interface that displays information for a group of patients. For example, a medical professional (e.g. a physician, nurse, nurse practitioner, dietician, or clinical educator) associated with a group of patients could use the second web interface to drive compliance for a disease-management program. Both web interfaces typically include multiple web pages that, in turn, feature both static and dynamic content, described in detail below.

The monitoring device typically measures: 1) heart rate; 2) systolic, diastolic, and pulse blood pressure; 3) pulse oximetry; and 4) cardiac ‘waveforms’ that can be further processed to determine arrhythmias, blood pressure load, and other cardiac properties. These properties can be measured daily as a one-time measurement, or quasi-continuously (e.g., every 30 seconds) during exercise. Preferably the monitoring device measures blood pressure without using a cuff in a matter of seconds, as described in detail below. This means patients can quickly and easily monitor blood pressure and other vital signs with minimal discomfort. With this device patients can easily measure their vital signs throughout the day (e.g., while at work), thereby generating a complete set of information, rather than just a single, isolated measurement. In addition, the monitoring device can collect weight and percent body fat from a bathroom scale (using, e.g., a wired or wireless link), and exercise-related properties, such as steps (using an internal pedometer circuit), calories burned (using sensor inputs and associated algorithms), and exercise time (using a simple clock).

In other embodiments, the monitoring device includes an interface (e.g., an RS232-based serial port, USB serial port, or wireless interface) to a personal computer. The wireless interface can include protocols such as Bluetooth™, 802.11, 802.15.4, and part-15. Typically, in this embodiment, the Internet-based system includes a software program that, when launched, collects vital sign and exercise information from the monitoring device. The Internet-based system may also link to ‘chat rooms’ or internal email systems that allow patients to communicate with one another.

The invention has many advantages, particularly because it provides a messaging engine that processes real-time information to, among other things, help a patient comply with a disease-management program, such as a personalized cardiovascular risk reduction program. The messaging engine analyses blood test, vital sign, exercise, and personal information, taken alone or combined, to generate personalized, patient-specific messages. Ultimately the Internet-based system, monitoring device, and messaging engine combine to form an interconnected, easy-to-use tool that can engage the patient in a disease-management program, encourage follow-on medical appointments, and build patient compliance. These factors, in turn, can help the patient lower their risk for certain medical conditions, such as CVD.

These and other advantages of the invention will be apparent from the following detailed description and from the claims.

### BRIEF DESCRIPTION OF THE DRAWINGS

FIG. 1 is a schematic drawing of an Internet-based system featuring a messaging engine that delivers personalized messages to a patient's email address, wireless device, and monitoring device;

FIG. 2 is a schematic view of a software program used in the messaging engine of FIG. 1 that processes patient, vital sign, exercise, and blood test information and, in response, deliver messages to the patient;

FIG. 3 is a detailed schematic view of the Internet-based system and messaging engine of FIG. 1;

FIG. 4 is a semi-schematic view of a monitoring device connecting through a USB port to a personal computer; and,

FIG. 5 shows a report resulting from a patient's blood test that generates blood test information for the messaging system of FIG. 1.

### DETAILED DESCRIPTION OF THE INVENTION

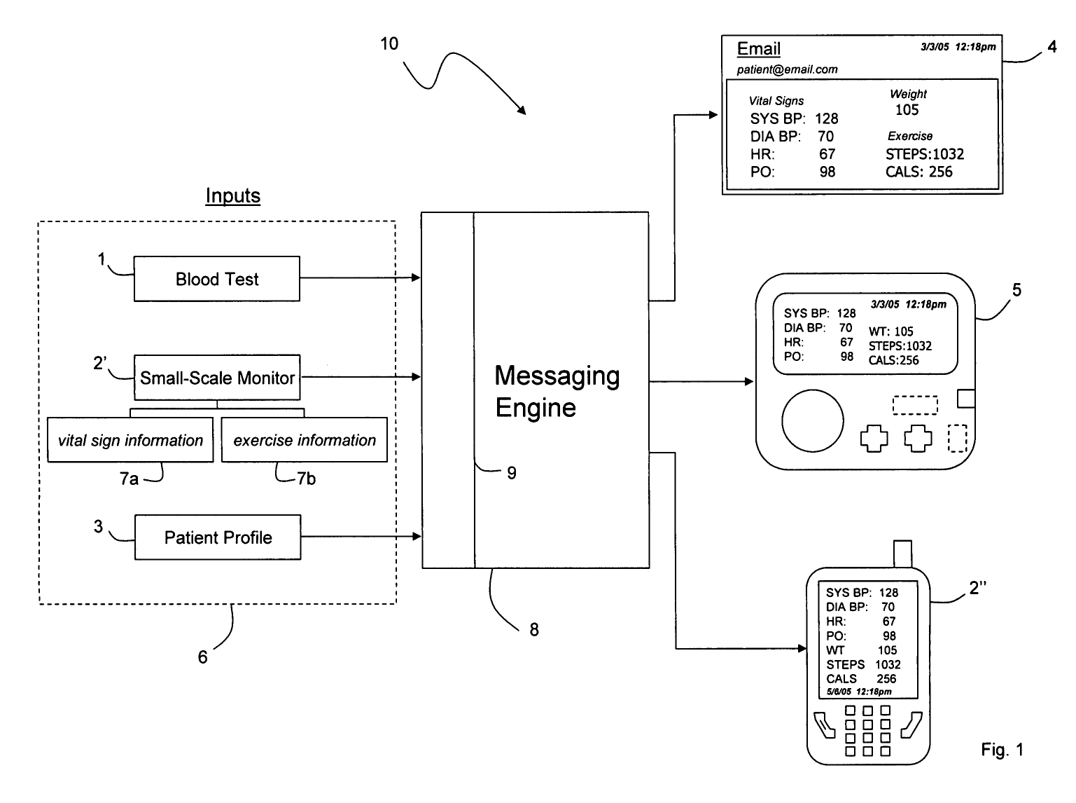

FIG. 1 shows a schematic drawing of an Internet-based system 10 featuring a messaging engine 8 that delivers personalized messages to a patient participating in a disease-management program. During operation, the messaging engine 8 receives information from multiple inputs 6, e.g., a blood test 1 and the monitoring device 2′. The inputs 6 can include a patient profile 3 (e.g., age, gender, medical history) that the patient supplies to the messaging engine 8 through a user interface. The monitoring device, described in detail below, typically includes systems that measure vital sign information 7a and exercise information 7b. A data-processing algorithm 9 running on the messaging engine 8 processes information from these inputs 6 to generate the personalized message, which the messaging engine 8 then sends to the patient's email address 4, wireless device 5, or to the monitoring device 2″. The personalized message is typically a text message that includes information describing the patient's vital signs, progress and content with regard to diet and exercise, and information describing a follow-on medical appointment. Its primary purposes are to engage the patient in the disease-management program, build patient compliance, motivate the patient to meet predetermined goals and milestones, and encourage the patient to schedule a follow-on medical appointment.

Several sample text messages that the messaging engine 8 may deliver to the patient are described below:

Patient: John Smith

Date: May 20, 2005

John—I noticed that over the last 2 weeks you lost 3 pounds and that your average blood pressure was 120 mmhg/70 mmhg. Great job! Here's a link to some recipes that may help you continue these positive trends. I look forward to seeing you for your follow-on blood test on June 15.

Chris Hall—Clinical Educator

Patient: John Smith

Date: May 20, 2005

John—your last cholesterol test measured your total cholesterol at 250 mg/dL; your HDL cholesterol (the ‘good cholesterol’) at 56 mg/dL; and your LDL cholesterol (the ‘bad cholesterol’) at 200 mg/dL. Since your total cholesterol and LDL cholesterol are above the values recommended by your physician, remember to continue the low-fat diet we planned during your last check-up, and exercise at least 5 times each week. I look forward to seeing you for your follow-on blood test on June 15.

Chris Hall—Clinical Educator

As described above, messages sent by the messaging engine 8 are meant to ‘touch’ the patient to help drive compliance in the disease-management program. To emphasize their personalized nature, the messages typically include blood test information collected from the blood test 1, and vital sign and exercise information collected by the monitoring device 2′. The data-processing algorithm 9 typically couples this information with text, content, and pre-determined verbiage to form the message. The personalized message, while generated by a computer hosting the Internet-based system 10, typically originates from the patient's clinical educator and includes the educator's contact information. The messaging engine 8 typically sends at least one personalized message per week.

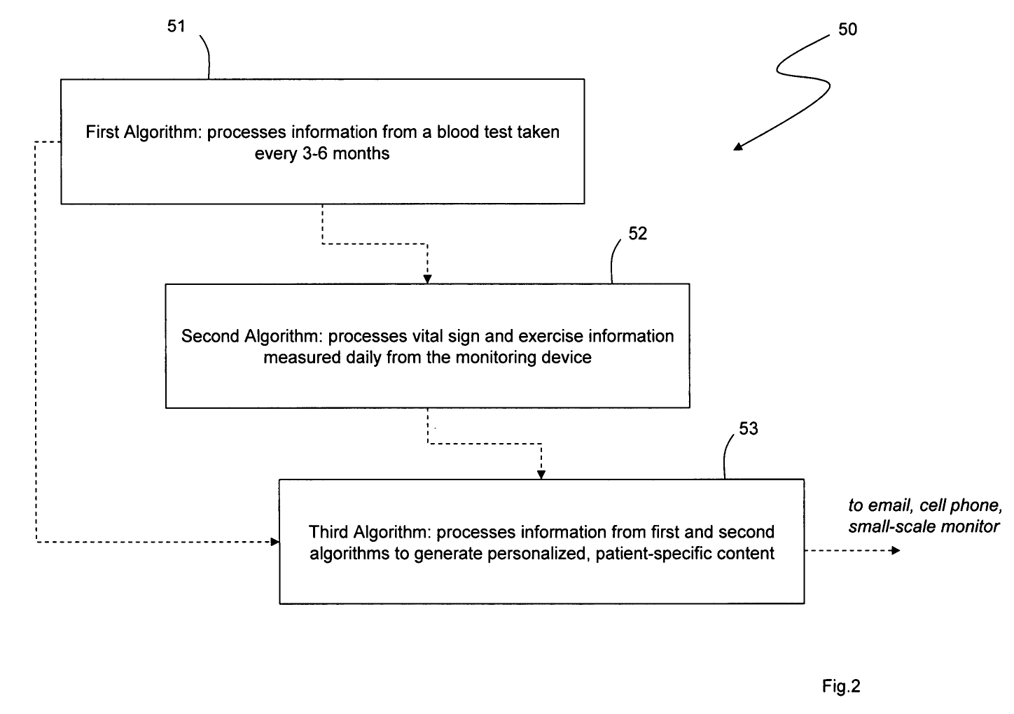

FIG. 2 shows a schematic drawing that illustrates a software program 50 used by the messaging engine in FIG. 1. Specifically, the software program 50 employs a first algorithm 51 that processes information from the patient's blood test. This information, which for example can originate from a GGE-based blood test taken every 3-6 months, typically describes amounts of blood-borne properties such as total cholesterol, LDL cholesterol (and subclass distribution), HDL cholesterol (and subclass distribution), triglycerides, Apo B-Particle, Apo B ultra particle, lipoprotein, Apo E genotype, fibrinogen, folate, HbA_{1c}, C-reactive protein, homocysteine, glucose, insulin, and chlamydia. In one embodiment, for example the first algorithm processes the patient's cholesterol values (e.g., total cholesterol, HDL cholesterol, LDL cholesterol, and sub-classes thereof) against pre-determined, medically approved levels to determine if these values are high, low, or normal.

A second algorithm 52 in the messaging engine 8 processes vital sign and exercise information from the monitoring device, which as described below is typically uploaded to the Internet-based system each day. This information typically includes vital sign information described in the pending patent application SMALL-SCALE, VITAL SIGNS MONITORING MONITOR, SYSTEM AND METHOD (U.S. Ser. No. 10/907440; filed Mar. 31, 2005), the contents of which are incorporated herein by reference. This information, for example, can include systolic, diastolic, and pulse blood pressure, heart rate, pulse oximetry, weight, percent body fat, calories burned, steps, and exercise time. Due to the relative high frequency that this information is loaded into the system, the second algorithm 52 typically generates statistics, such as time-dependent trends, average values, standard deviations, and minimum/maximum values. It also compares these values to pre-determined metrics to evaluate the patient's progress.

A third algorithm 53 processes the blood test information from the first algorithm 51, and vital sign and exercise information from the second algorithm 52, and in response generates patient-specific content for the personalized message. For example, the patient-specific content can include personalized verbiage, diet and exercise recommendations, recipes, program goals, progress toward goals, articles, educational content, links to web pages, or related content.

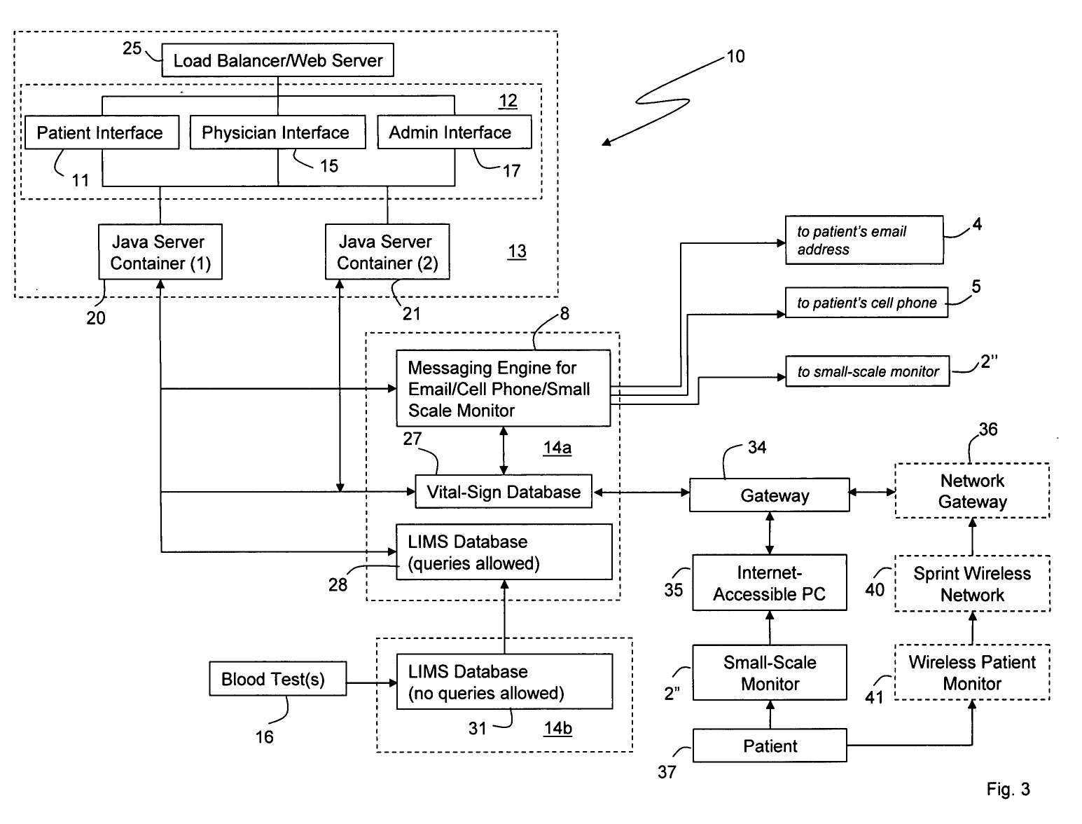

FIG. 3 shows in more detail the Internet-based system 10 that hosts the messaging engine 8. During operation, the messaging engine 8 sends messages described above in the form of emails sent to the patient's email address 4, text messages sent to the patient's cell phone 5 or to their small-scale vital sign monitor 2″. The messaging engine 8 is bi-directional and can also receive information from the patient, e.g. a confirmation of an appointment.

The Internet-based system 10 features a database layer 14a, 14b including a LIMS database 31 that automatically receives information directly from a blood test (e.g., one or more GGE test). The LIMS database 31 is typically located proximal to laboratory equipment used for the test. Typically no external database queries are performed on the LIMS database 31; its function is simply to store diagnostic information for each test. The LIMS database 31 periodically (e.g., once every 24 hours) uploads information to a matched LIMS database 28 hosted at an external site, e.g. a data center. Also hosted at the data center is a vital sign database 27 that collects vital sign information from a monitoring device 2′ associated with a patient 37. During operation, the monitoring device 2′ measures vital sign and exercise information from the patient as described in more detail below. The patient 37 then plugs the monitoring device 2′ into a USB port on an Internet-accessible personal computer 35, and logs into the patient interface 11. In response the patient interface 11 launches a software program that collects vital sign and exercise information stored in memory in the monitoring device 2′. The software program sends this information to an Internet-based gateway software program 34, which then formats the information and then sends it to the vital sign data database 27, where it is stored in memory for later processing.

The Internet-based system 10 additionally includes an administrative interface 17 that allows a user to, e.g., maintain and modify the application, query the database, and perform other administrative functions. The system 10 also includes a software load balancer/web server 25 that processes incoming http/https requests to regulate load placed on the site by outside users.

First 20 and second 21 Java server containers run software algorithms that process blood test information from the matched LIMS database 28 and vital sign information from the vital sign database 27 as described above to generate the personalized message. The personalized message can be conveyed to both the patient 37 and their physician through the patient 11 and physician 15 interfaces, and through the messaging engine 8 described above. Messages can originate from third parties other than the patient's clinical educator. These third parties include, for example, physician, nurse, nurse practioners, dietician, physical therapist, personal trainer, or other professional with access to the Internet-based system.

The Internet-based system 10 can also accept incoming vital sign information from a wireless patient monitor 41, such as that described in CUFFLESS BLOOD PRESSURE MONITOR AND ACCOMPANYING WIRELESS MOBILE MONITOR (U.S. Ser. No. 10/967,511; filed Oct. 18, 2004), the contents of which are incorporated by reference. In this embodiment, the wireless patient monitor 41 measures vital sign information from the patient 37, and transmits this information through a wireless network 40 to a network gateway software system 35. Using an Internet protocol, this system 35 sends the information to the gateway software system 34, which then stores it in the vital sign database 27 as described above.

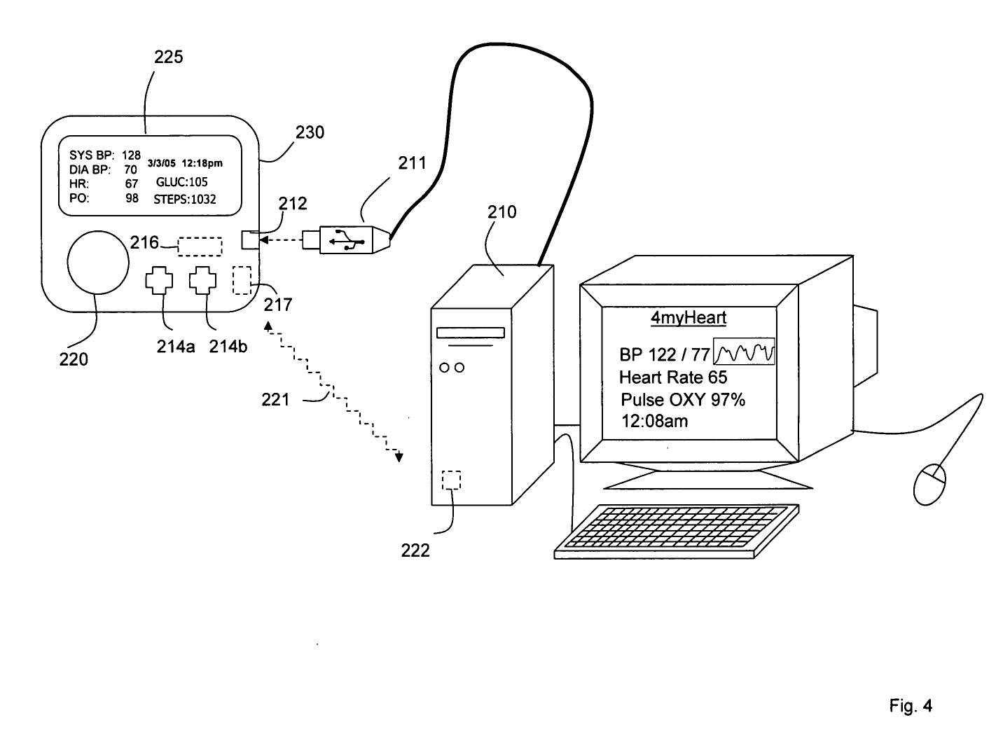

Referring to FIG. 4, the monitoring device 230 includes a USB port 212 through which it uploads and downloads information from an Internet-accessible personal computer 210 that connects to the Internet-based system. The USB port 212 typically connects to the personal computer 210 through a first cable 211. The monitoring device 230, which during use is typically attached to the patient's belt, features: i) an integrated, optical ‘pad sensor’ 220 that cufflessly measures blood pressure, pulse oximetry, and heart rate from a patient's finger as described in more detail below; and ii) an integrated pedometer circuit 216 that measures steps and, using an algorithm, calories burned. To receive information from external devices, the monitoring device 230 also includes a short-range wireless transceiver 217 that receives information such as body weight and percentage of body fat from an external scale. The wireless transceiver 217 can also connect through a wireless link 221 to the personal computer 210, which in this case includes a matched transceiver 222. The patient views information stored in the monitor using an LCD 225, and can interact with the monitor 230 (e.g., reset or reprogram it) using a series of buttons 214a, 214b.

Methods used by the monitoring device for measuring vital signs and particularly cuffless blood pressure are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No.; filed Sep. 13, 2004); 5) BLOOD PRESSURE MONITORING MONITOR FEATURING A CALIBRATION-BASED ANALYSIS (U.S. Ser. No. 10/967,610; filed Oct. 18, 2004); 6) PERSONAL COMPUTER-BASED VITAL SIGN MONITOR (U.S. Ser. No. 10/906,342; filed Feb. 15, 2005); 7) PATCH SENSOR FOR MEASURING BLOOD PRESSURE WITHOUT A CUFF (U.S. Ser. No. 10/906,315; filed Feb. 14, 2005 ); and 8) SMALL-SCALE, VITAL SIGNS MONITORING MONITOR, SYSTEM AND METHOD (U.S. Ser. No. 10/907440; filed Mar. 31, 2005).

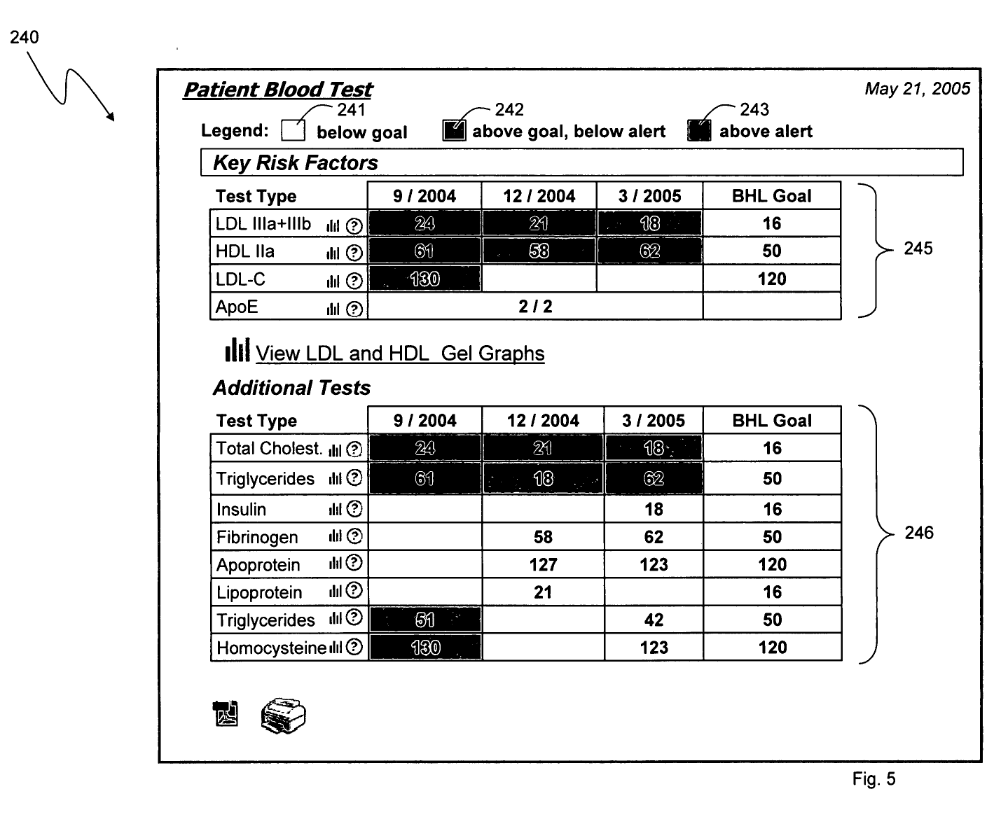

FIG. 5 shows a report 240 rendered by the Internet-based system that contains results taken from three blood tests conducted over a six-month period. The report 240, for example, could be included in a personalized message and rendered by the Internet-based system for both the patient and physician interfaces. It includes two primary fields 245, 246 describing a variety of parameters measured using a series of blood tests. Parameters in the first 245 and second 246 fields are compared to values recommended by a medical professional associated with the Internet-based system. Typically, parameters in the first 245 and second 246 fields are color-coded according to how they compare to the recommended values, and grouped into the following categories: ‘below goal’ 241; ‘above goal, below alert’ 242; and ‘above alert’ 243. ‘below goal’ 241 means a parameter has not met the goal established by the medical professional. Parameters that meet a goal, but fall under the status of an ‘alert’, are categorized as ‘above goal, below alert’ 242. A parameter that increases beyond that set by the medical professional falls into ‘above alert’ status 243.

Other embodiments are also within the scope of the invention. In particular, the web pages used to display information can take many different forms, as can the manner in which the data are displayed. Web pages are typically written in a computer language such as ‘HTML’ (hypertext mark-up language), and may also contain computer code written in languages such as Java and Java script for performing certain functions (e.g., sorting of names). The web pages are also associated with database software (provided by companies such as Oracle and Microsoft) that is used to store and access data. Equivalent versions of these computer languages and software can also be used. In general, the graphical content and functionality of the web pages may vary substantially from what is shown in the above-described figures. In addition, web pages may also be formatted using standard wireless access protocols (WAP) so that they can be accessed using wireless devices such as cellular telephones, personal digital assistants, and related devices.

Different web pages may be designed and accessed depending on the end-user. As described above, individual users have access to web pages that only their vital sign information (i.e., the patient interface), while organizations that support a large number of patients (e.g. hospitals) have access to web pages that contain information from a group of patients (i.e., the physician interface). Other interfaces can also be used with the web site, such as interfaces used for: hospitals, insurance companies, members of a particular company, clinical trials for pharmaceutical companies, and e-commerce purposes. Vital sign information displayed on these web pages, for example, can be sorted and analyzed depending on the patient's medical history, age, sex, medical condition, and geographic location.

The web pages also support a wide range of algorithms that can be used to analyze data once it is extracted from the data packets. For example, the above-mentioned text message or email can be sent out as an ‘alert’ in response to vital sign or blood test information indicating a medical condition that requires immediate attention. Alternatively, the message could be sent out when a parameter (e.g. blood pressure, heart rate) exceeded a predetermined value. In some cases, multiple parameters can be analyzed simultaneously to generate an alert message. In general, an alert message can be sent out after analyzing one or more data parameters using any type of algorithm. These algorithms range from the relatively simple (e.g., comparing blood pressure to a recommended value) to the complex (e.g., predictive medical diagnoses using ‘data mining’ techniques). In some cases data may be ‘fit’ using algorithms such as a linear or non-linear least-squares fitting algorithm. In general, any algorithm that processes data collected with the above-described method is within the scope of the invention.

In certain embodiments, the above-described can be used to characterize a wide range of maladies, such as diabetes, heart disease, congestive heart failure, sleep apnea and other sleep disorders, asthma, heart attack and other cardiac conditions, stroke, Alzheimer's disease, and hypertension.

Still other embodiments are within the scope of the following claims.

I claim as my invention:

1. A system for monitoring a patient, comprising:
a monitoring device comprising systems that monitor the patient's vital sign and exercise information;
an Internet-based system configured to receive, store, and display vital sign and exercise information from the monitoring device, and blood test information from an external blood test; and
a messaging system configured to process at least one of the vital sign, exercise, and blood test information, and in response send a personalized message to the patient.

2. The system of claim 1, wherein the messaging system is further configured to send a message to an email address, wireless device, or monitoring device.

3. The system of claim 2, wherein the messaging system is further configured to send at least one of a text message, SMS message, HTML-based message, or link to a web page.

4. The system of claim 1, wherein the messaging system is further configured to send a message that includes at least one of vital sign, exercise, and blood test information.

5. The system of claim 4, wherein the messaging system is further configured to send a message that additionally includes content describing at least one of diet recommendations, exercise recommendations, recipes, program goals, progress toward goals, articles, educational content, or related content.

6. The system of claim 4, wherein the messaging system is further configured to send a message that comprises a time-dependent trend in the patient's exercise behavior.

7. The system of claim 4, wherein the messaging system is further configured to send a message that includes a time-dependent trend in the patient's vital signs.

8. The system of claim 1, wherein the messaging system is further configured to receive messages from the patient.

9. The system of claim 8, wherein the messaging system is further configured to send a message that comprises a text field wherein the patient can enter a response.

10. The system of claim 9, further configured to include a software component configured to parse the response entered in the text field once the message is received.

11. The system of claim 2, wherein the wireless device is a cellular telephone or personal digital assistant.

12. The system of claim 1, wherein the vital sign information comprises at least one of systolic blood pressure, diastolic blood pressure, pulse blood pressure, heart rate, pulse oximetry, plethysmograph, weight, and percent body fat.

13. The system of claim 1, wherein the exercise information comprises at least one of exercise time, heart rate, steps, distance traveled, and calories burned.

14. The system of claim 1, wherein the blood test information describes the patient's cholesterol.

15. The system of claim 14, wherein the blood test information describes at least one sub-class of the patient's LDL cholesterol.

16. The system of claim 14, wherein the blood test information describes at least one sub-class of the patient's HDL cholesterol.

17. The system of claim 1, wherein the blood test information describes at least one of the following compounds: C-reactive protein, Apoliprotein A-1, Apoliprotein B, Apoliprotein E Isoforms, Fibrinogen, Folate, HbA_{1c}, Glucose, Insulin, Homocysteine, Lipoprotein (a), and Chlamydia.

18. The system of claim 1, wherein the Internet-based system comprises a website to display the blood test, cardiovascular and exercise information.

19. The system of claim 18, wherein the website comprises a first web interface that displays information for a single patient, and a second web interface that displays information for a group of patients.

20. A system for monitoring a patient, comprising:
a monitoring device comprising systems that monitor the patient's vital sign and exercise information;
an Internet-based system configured to receive, store, and display vital sign and exercise information from the monitoring device, and blood test information from an external blood test; and
a messaging system configured to process at least one of the vital sign, exercise, and blood test information, and in response send a personalized message to the patient, the message comprising at least one of the vital sign, exercise, and blood test information.

21. A system for monitoring a patient, comprising:
a monitoring device comprising systems that monitor the patient's vital sign and exercise information;
an Internet-based system configured to receive, store, and display vital sign and exercise from the monitoring device, and blood test information from an external blood test; and
a bi-directional messaging system configured to: 1) process at least one of the vital sign, exercise, and blood test information; 2) send a personalized message to the patient, the message comprising at least one of the vital sign, exercise, and blood test information; and 3) receive a message from the patient.
